(19) United States
(12) Patent Application Publication
(10) Pub. No.: US 2013/0065825 A1
(43) Pub. Date: Mar. 14, 2013

(54) Compositions and Methods for Delaying Senescence or Cell Death in Neurons

(71) Applicants: Steven J. Greco, Carlstadt, NJ (US); Nikolaos Tezapsidis, West Orange, NJ (US)

(72) Inventors: Steven J. Greco, Carlstadt, NJ (US); Nikolaos Tezapsidis, West Orange, NJ (US)

(73) Assignee: NEUROTEZ, INC., Bridgewater, NJ (US)

(21) Appl. No.: 13/613,030

(22) Filed: Sep. 13, 2012

Related U.S. Application Data
(60) Provisional application No. 61/534,500, filed on Sep. 14, 2011.

(51) Int. Cl. A61K 38/22; A61P 25/28; C12N 5/0793

(52) U.S. Cl. 514/5.8; 435/375

(57) ABSTRACT

The described invention provides methods and compositions for enhancing the enzymatic activity of Sirtuin family members by using Leptin, Leptin analogs, Leptin derivatives, or Leptin agonists. The described invention further provides compositions and methods for delaying senescence or cell death in neurons using the compositions.

### CROSS REFERENCE TO RELATED APPLICATIONS

This Application claims the benefit of priority to U.S. Provisional Application No. 61/534,500 (filed Sep. 14, 2011) entitled “COMPOSITIONS AND METHODS FOR DELAYING SENESCENCE OR CELL DEATH IN NEURONS,” the entire contents of which are incorporated by reference.

### STATEMENT OF GOVERNMENT FUNDING

The described invention was made with government support under Grant Number SBIR-1R43AG029670 awarded by the National Institute on Aging and the New Jersey Commission on Science and Technology. The government has certain rights in the invention.

### FIELD OF INVENTION

The described invention is related to compositions and methods for enhancing the enzymatic activity of Sirtuins in neurons or delaying senescence or cell death in neurons using Leptin, a Leptin analog or derivative, or a Leptin agonist.

### BACKGROUND

### Leptin and Alzheimer's Disease

Leptin is a pleiotropic hormone primarily secreted by adipocytes. Alzheimer's disease (AD) is marked by impaired brain metabolism with decreased glucose utilization in those regions, which often precede pathological changes. Recent epidemiological studies suggest that plasma Leptin is protective against AD. Specifically, elderly with plasma Leptin levels in the lowest quartile were found to be four times more likely to develop AD than those in the highest quartile.

Several pieces of evidence suggest that brain metabolic disturbances may precede the pathological cascades characteristic of AD. For example, functional neuroimaging studies, including 2-deoxy-2[(18)F]fluoro-D-glucose (FDG) positron emission topography (PET), have illustrated regional hypometabolism in the early AD brain; and that the pattern correlates with typical brain atrophy in AD (Kapogiannis, D. et al., Lancet Neurol., (2011), 10: 187-198; Sperling R. et al., Neuromolecular Med., (2010), 12: 27-43; Fouquet M. et al., Brain, (2009), 2058-2067; Mosconi L. et al., J. Nucl. Med. Mol. Imaging, (2009), 36: 811-822). Interestingly, pyramidal neurons of the hippocampus have particularly demanding energy needs, rendering the hippocampus a region more sensitive to states of metabolic distress (LaManna, J. et al., Brain Res., (1985), 326: 299-305).

Both genetic and environmental factors are likely contributors in this interconnection between brain metabolic state and disease. For example, carriers of one copy of the APOE4 gene, involved in lipid metabolism, are three- to four-fold more likely to develop AD than APOE3 carriers (Raber J. et al., Neurobiology of Aging, (2004), 25: 641-650). Further, carriers of the very long isotype of the TOMM40 gene polyT region among APOE3 carriers develop AD, on average, seven years earlier than carriers of the short isotype (Roses, A. et al., The Pharmacogenomics Journal, (2010), 10: 375-384).

Rodents fed high fat/caloric diets with limited exercise demonstrate impaired learning and memory performance compared to similar animals on lower energy diets. In humans, a large longitudinal analysis showed a significant correlation between central obesity in midlife and an increased risk of dementia independent of diabetes and cardiovascular co-morbidities later in life (Mark. R. et al., J. Neurosci., (1997), 71: 1046-1054).

Once AD pathological cascades are initiated because of these metabolic disturbances, these could further and cyclically exacerbate hypometabolic states regionally. Amyloid beta (Aβ) oligomers induce oxidative stress (Mark, R. et al., J. Neurosci., (1997), 17:1046-1054), while activation of Glycogen Synthase Kinase-3β (GSK-3β) (promoted by Aβ and inhibited by Leptin), one of the many kinases that can phosphorylate tau, leads to decreased mitochondrial membrane potential and ATP production (Shukkur, E. et al., Hum. Mol. Genet., 2006, 15: 2752-2762). Studies utilizing animal models of aging and AD have shown that achieving optimal energy balance (i.e. through feeding and exercise) can improve cognitive function and prevent an age-related decline in learning (Ingram, D. et al., J. Gerontol, (1987), 42: 78-81; Nichol, K. et al., Alzheimers Dement, (2009), 5: 287-294).

Leptin, primarily secreted from adipocytes, can function as a modulator of energy metabolism (Morton, G. et al., J Physiol, (2007), 583: 437-443). Within the arcuate nucleus of the hypothalamus, Leptin acts on neuropeptide Y/agouti-related peptide (NPY/AgRP) and pro-opiomelanocortin (POMC) neurons to regulate food intake, energy expenditure, and hepatic glucose production (Schwartz, M. et al., Science, (2005), 307: 375-379). However, other larger regions in the brain, including the cortex and the hippocampus, are also known to express high levels of the Ob—Rb, the long isoform of Leptin receptor, which are known to transduce signaling (Huang, X. et al., Neuroreport, (1996), 7: 2635-2638). Peripherally, Leptin acts directly on fat and skeletal muscle to stimulate fatty acid oxidation, to increase glucose uptake, and to inhibit lipogenesis (Long, Y. et al., J Clin Invest, (2006), 116: 1776-1783). In both settings, Leptin production is stimulated by a positive energy balance and acts to restore energy homeostasis through suppressing anabolic and boosting catabolic pathways.

To date, a number of reports have shown that there is a positive correlation between reduced levels of circulating Leptin and AD risk (Olsson, T. et al., Biol. Psychiatry, (1998), 44: 374-376; Power, D. et al., Dement. Geriatr. Cogn. Disord., (2001), 12:167-170), severity of dementia (Ray, S. et al., WO 2005/052592 A2, Satoris, Inc., 2005), and cognitive decline (Holden, K. et al. American Academy of Neurology, 58th Annual Meeting, San Diego, 2006, pp. 541.006; Holden K. et al.). Most notably, a study involving 785 cognitively-normal elderly followed for a median of 8.3 years showed that those with plasma Leptin levels in the lowest quartile at baseline were at four times greater risk for developing AD than those in the highest quartile (Lieb, W. et al., Journal of the American Medical Association, (2009), 302: 2565-2572). At the physiological level, it is known that there is a high concentration of Leptin receptors in the hippocampus (Huang, X. et al., Neuroreport, (1996), 7: 2635-2638), which are functional, and that direct injection of Leptin in that region can improve memory processing and modulate long term potentiation and synaptic plasticity (Harvey, J. et al., Biochem Soc Trans, (2005), 33, 1029-1032). Leptin administration improves memory in SAMP-8 mice, an accelerated senescence rodent model that develops amyloid plaques (Farr, S. et al., Peptides, (2006), 27:1420-1425). Moreover, the diabetic/obese db/db mice, which lack a functional Leptin receptor, exhibit cognitive impairment and impaired synaptic function and neurogenesis (Stranahan, A. et al., Nat Neurosci, (2008), 11: 309-317). Interestingly, it has been suggested that one of Leptin's roles could involve the prevention of excess accumulation of lipids in non-adipocytes, including neurons, which could be poisoning (Unger, R. et al., Biochimie (2005), 87: 57-64).

### Leptin and AMPK

Leptin reduces tau phosphorylation and Aβ production in neuronal cells and transgenic mice models of AD. Leptin's effects in vitro were dependent on activation of the cellular energy sensor, AMP-activated protein kinase (AMPK) (Greco, S. et al., Biochem. Biophys. Res. Commun. (2008), 376: 536-541). AMPK is ubiquitously expressed throughout the body and is activated in states of low cellular energy by an elevated AMP/ATP ratio (Winder, W. et al., Am. J. Physiol. (1999), 277: E1-10). Besides ATP, the only other small molecule in cells that indicates energy status is NAD+, which is necessary for activation of a family of evolutionarily conserved energy sensors, the Sirtuins (SIRT) (Imai, S. et al., Nature (2000), 403: 795-800).

### Sirtuins (SIRT)

The Sirtuins are histone deacetylases that play important roles in a number of physiological processes, including stress resistance (Cohen, H. et al., Science (2004), 305: 390-392), replicative senescence (Chua, K. et al., Cell Metabolism, (2005), 2: 67-76), aging and differentiation (Blander, G. et al., Annual Review of Biochemistry, (2004), 73: 417-435). Notably SIRT1 has been associated with the anti-aging effects of caloric restriction and, most recently, inhibition of amyloidogenic pathways in laboratory models of AD (Chen, D. et al., Science, (2005), 310: 1641; Donmez, G. et al., Cell, (2010), 142: 320-332; Bonda, D. et al., Lancet Neurology (2011), 10: 275-279). Additionally, caloric restriction has been shown to indirectly activate SIRT1 through a linear pathway involving AMPK (Fulco, M. et al., Developmental Cell, (2008), 14: 661-673).

While a high density of functional Leptin receptors have been reported to be expressed in the hippocampus and other cortical regions of the brain, the physiological significance has not been explored extensively.

### SUMMARY OF THE INVENTION

According to one aspect, the described invention provides a method for enhancing an enzymatic activity of at least one family member of Sirtuins (SIRT) in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to enhance the enzymatic activity of at least one family member of Sirtuins (SIRT) in the neuronal cell population.

According to one embodiment of the method, the Leptin analog or derivative is a functional analog of Leptin that is capable of binding to a Leptin receptor (OB—R) and of inducing a signal transduction pathway via the Leptin receptor (OB—R) inside the cell. According to another embodiment, the Leptin analog or derivative is selected from the group consisting of adiponectin, LY396623, Metreleptin, a murine Leptin analog, pegylated Leptin, methionyl human Leptin, Resistin, and a combination thereof. According to another embodiment, the Leptin analog or derivative is a peptide or polypeptide in which at least one amino acid residue of Leptin has been replaced with at least one non-naturally occurring amino acid selected from the group consisting of beta-alanine, alpha amino butyric acid, gamma-amino butyric acid, alpha-isobutryic acid, norvaline, norleucine, epsilon-lysine, ornithine, homoserine, and hydroxyproline. According to another embodiment, the Leptin agonist is an activator of AMP-activated protein kinase (AMPK). According to another embodiment, the activator of AMP-activated protein kinase (AMPK) is selected from the group consisting of phenformin, 5-aminoimidazole-4-carboxamide riboside (AICAR), metformin, rosiglitazone, and a combination thereof. According to another embodiment, contacting the neuronal cell population comprises administering the composition comprising the effective amount of the Leptin, the Leptin analog or derivative, or the Leptin agonist, and a carrier to a mammal in vivo. According to another embodiment, contacting the neuronal cell population with the composition enhances an enzymatic activity of at least one family member of Sirtuins or AMP-activated Protein Kinase (AMPK) in the neuronal cell population compared to a control neuron population without treatment. According to another embodiment, the family member of Sirtuins is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof. According to another embodiment, the enzymatic activity of total Sirtuins in the neuronal cell population treated with the composition is at least two times greater than the enzymatic activity of total Sirtuins in a control neuron population without treatment. According to another embodiment, the neuronal cell population comprises a neuronal population of a central nervous system that expresses Obese Receptor (Ob—R). According to another embodiment, the Obese Receptor is Obese receptor—Rb (Ob—Rb). According to another embodiment, the neuronal cell population comprises a hippocampal neuron population. According to another embodiment, the neuronal cell population comprises a cortical neuron population. According to another embodiment, the neuronal cell population comprises a Purkinje neuron population. According to another embodiment, the neuronal cell population comprises a basal ganglia neuron population. According to another embodiment, the neuronal cell population comprises an olfactory neuron population. According to another embodiment, the neuronal cell population comprises a dopaminergic neuron population. According to another embodiment, the neuronal cell population comprises a noradrenergic neuron population. According to another embodiment, the neuronal cell population comprises a motor neuron population. According to another embodiment, the motor neuron population comprises a spinal motor neuron population. According to another embodiment, the neuronal cell population comprises a sensory neuron population. According to another embodiment, the neuronal cell population comprises an interneuron population. According to another embodiment, the neuronal cell population comprises a neuronal population of peripheral nervous system.

According to another aspect, the described invention provides a method for delaying senescence or cell death of a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is to effective to delay senescence or cell death of the neuronal cell population.

According to one embodiment of the method, the Leptin analog or derivative is a functional analog of Leptin, which are capable of binding to a Leptin receptor (OB—R) and inducing a signal transduction pathway via the Leptin receptor (OB—R) inside the cell. According to another embodiment, the Leptin analog or derivative is selected from the group consisting of adiponectin, LY396623, Metreleptin, a murine Leptin analog, pegylated Leptin, methionyl human Leptin, Resistin, and a combination thereof. According to another embodiment, the Leptin analog or derivative is a peptide or polypeptide in which at least one amino acid residue of Leptin has been replaced with non-naturally occurring amino acids selected from the group consisting of beta-alanine, alpha amino butyric acid, gamma-amino butyric acid, alpha-isobutryic acid, norvaline, norleucine, epsilon-lysine, ornithine, homoserine, and hydroxyproline. According to another embodiment, the Leptin agonist is an activator of AMP-activated protein kinase (AMPK). According to another embodiment, the activator of AMP-activated protein kinase (AMPK) is selected from the group consisting of phenformin, 5-aminoimidazole-4-carboxamide riboside (AICAR), metformin, rosiglitazone, and a combination thereof. According to another embodiment, contacting the neuronal cell population comprises administering the composition comprising the effective amount of the Leptin, the Leptin analog or derivative, or the Leptin agonist, and the carrier to a mammal in vivo. According to another embodiment, contacting the neuronal cell population with the composition enhances an enzymatic activity of at least one family member of Sirtuins or AMP-activated Protein Kinase (AMPK) in the neuronal cell population compared to a control neuron population without treatment. According to another embodiment, the family member of Sirtuins is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof. According to another embodiment, an enzymatic activity of total Sirtuins in the neuronal cell population treated with the composition is at least two times greater than the enzymatic activity of total Sirtuins in a control neuron population without treatment. According to another embodiment, the enzymatic activity of the AMP-activated Protein Kinase (AMPK) in the neuronal cell population treated with the composition is at least two times greater than the enzymatic activity of the AMP-activated Protein Kinase (AMPK) in a control neuron population without treatment. According to another embodiment, the neuronal cell population comprises a neuronal population of central nervous system that expresses Obese Receptor (OB—R). According to another embodiment, the Obese Receptor is Obese Receptor—Rb (Ob—Rb). According to another embodiment, the neuronal cell population comprises a hippocampal neuron population. According to another embodiment, the neuronal cell population comprises a cortical neuron population. According to another embodiment, the neuronal cell population comprises a Purkinje neuron population. According to another embodiment, the neuronal cell population comprises a basal ganglia neuron population. According to another embodiment, the neuronal cell population comprises an olfactory neuron population. According to another embodiment, the neuronal cell population comprises a dopaminergic neuron population. According to another embodiment, the neuronal cell population comprises a noradrenergic neuron population. According to another embodiment, the neuronal cell population comprises a motor neuron population. According to another embodiment, the motor neuron population comprises a spinal motor neuron population. According to another embodiment, the neuronal cell population comprises a sensory neuron population. According to another embodiment, the neuronal cell population comprises an interneuron population. According to another embodiment, the neuronal cell population comprises a neuronal population of peripheral nervous system.

### BRIEF DESCRIPTION OF THE DRAWINGS

FIG. 1 shows the effect of Leptin on 5′-Adenosine Monophosphate (AMP)-Activated Protein Kinase (AMPK) and Sirtuin (SIRT) activity in RA-SY5Y cells. Cells were treated for 6 h with or without Leptin (500 ng/mL) in the presence or absence of the AMPK inhibitor, compound C (20 μM), or the SIRT inhibitor, nicotinamide (5 mM), and (A) AMPK or (B) SIRT activity measured. All activity values were normalized to total protein, n=3; *p<0.05 vs. no Leptin; **p<0.05 vs. control

FIG. 2 shows that inhibition of AMPK and SIRT negates Leptin's effect on tau phosphorylation in RA-SY5Y cells. (A) Cells were treated for 6 h with or without Leptin (500 ng/mL) in the presence or absence of nicotinamide (5 mM) or compound C (20 μM), and cell lysates probed for phosphorylated tau¹⁸¹ by immunoblot (inset). Blots were normalized by stripping and reprobing for total tau and densitometry analyses performed, n=3. Extracts from A were assayed by ELISA to quantify total and phosphorylated (B) tau²³¹ and (C) tau³⁹⁶. All concentration values were normalized to total tau, n=3; *p<0.05 vs. no Leptin; **p<0.05 vs. control

FIG. 3 shows that inhibition of AMPK and SIRT negates Leptin's effect on Aβ₍₁₋₄₀₎ in RA-SY5Y cells. Cells were treated overnight with or without Leptin (500 ng/mL) in the presence or absence of nicotinamide (5 mM) or compound C (20 μM), and culture media collected for determination of Aβ₍₁₋₄₀₎ levels by ELISA. Results were normalized to total protein and presented as a percentage relative to vehicle control, n=3; *p<0.05 vs. no Leptin.

### DETAILED DESCRIPTION OF THE INVENTION

Glossary

The term “active” refers to the ingredient, component or constituent of the compositions of the described invention responsible for the intended therapeutic effect.

The term “administer” as used herein means to give or to apply. The term “administering” as used herein includes in vivo administration, as well as administration directly to tissue ex vivo.

The terms “amino acid residue” or “amino acid” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids. The amino acids may be L- or D-amino acids. An amino acid may be replaced by a synthetic amino acid, which is altered so as to increase the half-life of the peptide, increase the potency of the peptide, or increase the bioavailability of the peptide.

The single letter designation for amino acids is used predominately herein. As is well known by one of skill in the art, such single letter designations are as follows:

A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine.

The following represents groups of amino acids that are conservative substitutions for one another: 1) Alanine (A), Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.”

Derivatives can include chemical modifications of the Leptin peptide, such as akylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those Leptin peptides that contain one or more naturally occurring amino acid derivative of the twenty standard amino acids, for example, 4-hydroxyproline, 5-hydroxylysine, 3-methylhistidine, homoserine, ornithine or carboxyglutamiate, and can include amino acids that are not linked by peptide bonds. Such peptide derivatives can be incorporated during synthesis of the Leptin peptide, or a Leptin peptide can be modified by well known chemical modification methods (see, e.g., Glazer et al., Chemical Modification of Proteins, Selected Methods and Analytical Procedures, Elsevier Biomedical Press, New York (1975)).

The term “Leptin analog” or “Leptin derivative” as used herein refers to a functional analog of Leptin, which is capable of binding to a Leptin receptor (OB—R) and is able to induce a downstream signal transduction pathway inside a cell. The functional analog of Leptin comprises a functional domain of the Leptin protein, alone or in combination with another molecule, which can produce a biological effect, namely the effect of activating a signal transduction pathway mediated by a Leptin receptor (OB—R), inside a cell. According to some embodiments, the term “Leptin analog or Leptin derivative” refers to a compound containing a non-peptidic structural element capable of mimicking the biological action(s) of the Leptin peptide, for example, a peptidomimetic, which incorporates the portion of Leptin mediating an enzymatic activity and has a size small enough to penetrate the blood-brain barrier.

The term “Leptin agonist” as used herein refers to a compound capable of activating the Leptin receptor and/or its downstream effectors, such as AMP-activated protein kinase (AMPK), inside a cell. Examples of AMPK activator include, but are not limited to, phenformin, 5-aminoimidazole-4-carboxamide riboside (AICAR), metformin, and rosiglitazone (King et al. Biochem. Pharmacol. 71:1637-47 (2006)).

The Leptin receptor (OB—R), a member of the class I cytokine receptor superfamily, has at least six isoforms as a result of alternative splicing. All isoforms of OB—R share an identical extracellular ligand-binding domain. Leptin's functional receptor (OB—Rb), the b isoform, is expressed not only in the hypothalamus, where it regulates energy homeostasis and neuroendocrine function, but also in other brain regions and in the periphery, including all cell types of innate and adaptive immunity. The full-length b isoform (OB—Rb) lacks intrinsic tyrosine kinase activity and is involved in several downstream signal transduction pathways.

The term “agonist” as used herein refers to a chemical substance capable of activating a receptor to induce a full or partial pharmacological response. Receptors can be activated by either endogenous or exogenous agonists and can be inhibited by antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist.

The terms “amyloid peptide”, “amyloid p peptide”, and “Aβ” are used interchangeably herein to refer to the family of peptides generated through proteolytic processing of amyloid precursor protein (APP).

The term “antagonist” as used herein refers to a substance that counteracts the effects of another substance.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κ.

In mammals, there are five classes of antibodies, IgA, IgD, IgE, IgG, and IgM, each with its own class of heavy chain—α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “carrier” as used herein describes a material that does not cause significant irritation to an organism and does not abrogate the biological activity and properties of the compound of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “cell culture” as used herein refers to establishment and maintenance of cultures derived from dispersed cells taken from original tissues, primary culture, or from a cell line or cell strain.

The term “cell line” as used herein refers to a population of immortalized cells that have undergone transformation and can be passed indefinitely in culture.

The term “cell passage” as used herein refers to the splitting (dilution) and subsequent redistribution of a monolayer or cell suspension into culture vessels containing fresh media.

The term “cell strain” as used herein refers to cells that can be passed repeatedly but only for a limited number of passages.

The term “contact” and its various grammatical forms as used herein refers to a state or condition of touching or of immediate or local proximity. Contacting a composition to a target destination, such as, but not limited to, an organ, a tissue, a cell, or a tumor, may occur by any means of administration known to the skilled artisan.

The term “polyclonal” refers to derived from many clones; “oligoclonal” refers to derived from a few clones; “monoclonal” refers to derived from one clone.

The term “component” as used herein refers to a constituent part, element or ingredient.

The term “condition”, as used herein, refers to a variety of health states and is meant to include disorders or diseases caused by any underlying mechanism or disorder, injury, and the promotion of healthy tissues and organs.

The term “differentiation” as used herein refers to the process by which a less specialized cell becomes a more specialized cell type.

The term “drug” as used herein refers to a therapeutic agent or any substance, other than food, used in the prevention, diagnosis, alleviation, treatment, or cure of disease.

The term “enhancing ” the biological activity, function, health, or condition of an organism as used herein refers to the process of augmenting, fortifying, strengthening, or improving.

The terms “epitope” and “antigenic determinant” are used interchangeably herein to refer to the site on a molecule that an antigen combining site (ACS) recognizes and to which that antibody binds/attaches itself. In some embodiments, an epitope may be an antigenic determinant/antigen binding site on a kinase inhibiting peptide. The epitope may be primary, secondary, or tertiary-sequence related.

The term “functional equivalent” or “functional homolog” as used herein refers to substances, molecules, peptides, polypeptides, or proteins having similar or identical effects or use. A substance, molecule, peptide, polypeptide, or protein, which is functionally equivalent to Leptin, for example, may have a biological activity (e g , inhibitory activity, kinetic parameters, and a cofactor dependent activity) that is substantially similar or identical to Leptin.

The term “substantially similar to Leptin” as used herein means that a biological activity of a substance, a molecule, a peptide, a polypeptide, or a protein is at least 70%, 75%, 80%, 85%, 90%, 95%,96%, 97%, 98%, or at least 99% similar to the biological activity of Leptin.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “AMP-Activated Protein Kinase (AMPK) inhibitor” or “AMPK inhibitor” as used herein refers to a compound that inhibits the kinase activity of 5′-Adenosine Monophosphate (AMP)-Activated Protein Kinase (AMPK) and activation of its downstream signal transduction pathway. Examples of AMPK inhibitors include, but are not limited to, compound C (6-[4-(2-Piperidin-1-yl-ethoxy)-phenyl]-3-pyridin-4-yl-pyrrazolo[1,5,-α]pyrimidine) and Adenine-9-β-D-arabinofuranoside A (Ara A).

The term “Sirtuin inhibitor” or “SIRT inhibitor” as used herein refers to a compound that inhibits deacetylase activity or mono-ribosyl transferase activity of Sirtuin (SIRT). For example, a SIRT inhibitor may inhibit one or more activities of a mammalian Sirtuins, e.g., SIRT1, SIRT2, SIRT3, SIRT4, SIRT5, SIRT6, and SIRT7. Examples of a SIRT inhibitor include, but are not limited to, sirtinol, splitomycin, and nicotinamide.

A “pharmaceutical composition” is one that is employed to prevent, reduce in intensity, cure, or otherwise treat a target condition, syndrome, disorder or disease that has undergone federal regulatory review.

The term “primary culture” as used herein refers to cells resulting from the seeding of dissociated tissues. Primary cultures often lose their phenotype and genotypes within several passages. Most primary cell cultures have limited lifespan, with the exception of some derived from tumors.

The term “recombinant” as used herein refers to a substance produced by genetic engineering.

The term “reduced” or “to reduce” as used herein refer to a diminution, a decrease, an attenuation or abatement of the degree, intensity, extent, size, amount, density or number.

The term “similar” is used interchangeably with the terms analogous, comparable, or resembling, meaning having traits or characteristics in common.

The term “total Sirtuins” as used herein refers to total Sirtuin proteins expressed by a given cell type, for example, without limitation, Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

The term “susceptible” as used herein refers to a member of a population at risk.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The term “therapeutic agent” as used herein refers to a drug, molecule, nucleic acid, protein, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “effective amount”, “therapeutically effective amount” or an “amount effective” of one or more of the active agents is an amount that is sufficient to provide a described biological effect or the intended benefit of treatment. An effective amount of the active agents that can be employed ranges from generally 0.1 mg/kg body weight and about 50 mg/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

I. Methods for Enhancing an Enzymatic Activity of Sirtuin Family Members in Neurons

According to one aspect, the described invention provides a method for enhancing an enzymatic activity of at least one family member of Sirtuins (SIRT) in a neuronal cell population, comprising contacting the neuronal cell population with a composition containing an effective amount of Leptin, a Leptin analog or derivative, or a Leptin agonist, and a carrier.

According to some embodiments, the Leptin analog or derivative includes functional analogs of Leptin, which are capable of binding to a Leptin receptor (OB—R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, for example, orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means, such as, but not limited to, injection, implantation, grafting, or topical application. The term “topical” as used herein refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

According to another embodiment, the composition is administered parenterally. The term “parenteral” as used herein refers to introduction into the body by way of an injection (i.e., administration by injection), including, for example, subcutaneous injection (i.e., an injection beneath the skin), intramuscular injection(i.e., an injection into a muscle), intravenous injection (i.e., an injection into a vein), intrathecal injection (i.e., an injection into the space around the spinal cord or under the arachnoid membrane of the brain), intrasternal injection (i.e., injection into the sternum (a long flat bone that is situated along the ventral midline of the thorax and articulates with the ribs)), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

According to another embodiment, administering can be performed once, a plurality of times, and/or over one or more extended periods either as individual unit doses or in the form of a treatment regimen comprising multiple unit doses of multiple drugs and/or substances.

According to another embodiment, the family member of Sirtuin is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

According to another embodiment, the enzymatic activity of total Sirtuins in the neuronal cell population treated with the composition is at least two times greater than the enzymatic activity of total Sirtuins in a control neuron population without treatment.

According to another embodiment, the neuronal cell population comprises a neuronal population of the central nervous system, which expresses a Leptin receptor (OB—R). According to another embodiment, the receptor is Obese Receptor—Rb (Ob—Rb).

According to another embodiment, the neuronal cell population includes, but is not limited to, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuron population of the peripheral nervous system.

II. Methods for Delaying Senescence or Cell Death in Neuronal Cells

According to another aspect, the described invention provides a method for delaying senescence or cell death in a neuronal cell population, the method comprising contacting the neuronal cell population with a composition containing an effective amount of Leptin, a Leptin analog or derivative, or a Leptin agonist, and a carrier.

According to some embodiments, the Leptin analog or derivative includes functional analogs of Leptin, which are capable of binding to a Leptin receptor (OB—R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, e.g., orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, or topical application.

According to another embodiment, the family member of Sirtuin includes Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

According to another embodiment, the enzymatic activity of total Sirtuins in the neuronal cell population treated with the composition is least two times greater than the enzymatic activity of total Sirtuins in a control neuronal population without treatment.

According to another embodiment, the enzymatic activity of the 5′-Adenosine monophosphate (AMP)-activated Protein Kinase (AMPK) in the neuronal cell population treated with the composition is at least two times greater than the enzymatic activity of the 5′-Adenosine monophosphate (AMP)-activated Protein Kinase (AMPK) in an untreated control neuron population.

According to another embodiment, the neuronal cell population is a neuronal population of the central nervous system, which expresses a Leptin receptor (Ob—R). According to another embodiment, the receptor is Obese Receptor Rb (Ob—Rb).

According to another embodiment, the neuronal cell population includes, but is not limited to, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuronal population of peripheral nervous system.

The therapeutic agents in the compositions are delivered in therapeutically effective amounts. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular therapeutic agent(s) being administered, the size of the subject, or the severity of the disease or condition. One of ordinary skill in the art may determine empirically the effective amount of a particular therapeutic agent(s) without necessitating undue experimentation. It generally is preferred that a maximum tolerated dose be used, that is, the highest safe dose according to some medical judgment. The terms “dose” and “dosage” are used interchangeably herein.

For any Leptin, Leptin analogs or derivatives, or Leptin agonists, described herein, the therapeutically effective amount may be initially determined from preliminary in vitro studies and/or animal models. A therapeutically effective dose also may be determined from human data for therapeutic agent(s) which have been tested in humans and for compounds which are known to exhibit similar pharmacological activities, such as other related active agents. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

According to another embodiment, the therapeutically effective amount of the composition is of an amount from about 0.000001 mg/kg body weight to about 10 g/kg body weight. According to another embodiment, the therapeutically effective amount of the composition is of an amount from about 0.000002 mg/kg body weight to about 10 g/kg body weight. According to another embodiment, the therapeutically effective amount of the composition is of an amount from about 0.000003 mg/kg body weight to about 10 g/kg body weight. According to another embodiment, the therapeutically effective amount of the composition is of an amount from about 0.000004 mg/kg body weight to about 10 g/kg body weight. According to another embodiment, the therapeutically effective amount of the composition is of an amount from about 0.000005 mg/kg body weight to about 10 g/kg body weight. According to another embodiment, the therapeutically effective amount of the composition is of an amount from about 0.000006 mg/kg body weight to about 10 g/kg body weight. According to another embodiment, the therapeutically effective amount of the composition is of an amount from about 0.000007 mg/kg body weight to about 10 g/kg body weight. According to another embodiment, the therapeutically effective amount of the composition is of an amount from about 0.000008 mg/kg body weight to about 10 g/kg body weight. According to another embodiment, the therapeutically effective amount of the composition is of an amount from about 0.000009 mg/kg body weight to about 10 g/kg body weight. According to another embodiment, the therapeutically effective amount of the composition is of an amount from about 0.00001 mg/kg body weight to about 10 g/kg body weight. According to another embodiment, the therapeutically effective amount of the composition is of an amount from about 0.00002 mg/kg body weight to about 10 g/kg body weight. According to another embodiment, the therapeutically effective amount of the composition is of an amount from about 0.0003 mg/kg body weight to about 10 g/kg body weight. According to another embodiment, the therapeutically effective amount of the composition is of an amount from about 0.00004 mg/kg body weight to about 10 g/kg body weight. According to another embodiment, the therapeutically effective amount of the composition is of an amount from about 0.00005 mg/kg body weight to about 10 g/kg body weight. According to another embodiment, the therapeutically effective amount of the composition is of an amount from about 0.00006 mg/kg body weight to about 10 g/kg body weight. According to another embodiment, the therapeutically effective amount of the composition is of an amount from about 0.00007 mg/kg body weight to about 10 g/kg body weight. According to another embodiment, the therapeutically effective amount of the composition is of an amount from about 0.00008 mg/kg body weight to about 10 g/kg body weight. According to another embodiment, the therapeutically effective amount of the composition is of an amount from about 0.00009 mg/kg body weight to about 10 g/kg body weight. According to another embodiment, the therapeutically effective amount of the composition is of an amount from about 0.0001 mg/kg body weight to about 10 g/kg body weight. According to another embodiment, the therapeutically effective amount of the composition is of an amount of about 0.0005 mg/kg body weight. According to another embodiment, the therapeutically effective amount of the composition is of an amount of about 0.001 mg/kg body weight. According to another embodiment, the therapeutically effective amount of the composition is of an amount of about 0.005 mg/kg body weight. According to another embodiment, the therapeutically effective amount of the composition is of an amount of about 0.01 mg/kg body weight. According to another embodiment, the therapeutically effective amount of the composition is of an amount of about 0.1 mg/kg body weight. According to another embodiment, the therapeutically effective amount of the composition is of an amount of about 1 mg/kg body weight. According to another embodiment, the therapeutically effective amount of the composition is of an amount of about 10 mg/kg body weight. According to another embodiment, the therapeutically effective amount of the composition is of an amount of about 20 mg/kg body weight. According to another embodiment, the therapeutically effective amount of the composition is of an amount of about 30 mg/kg body weight. According to another embodiment, the therapeutically effective amount of the composition is of an amount of about 40 mg/kg body weight. According to another embodiment, the therapeutically effective amount of the composition is of an amount of about 50 mg/kg body weight. According to another embodiment, the therapeutically effective amount of the composition is of an amount of about 60 mg/kg body weight. According to another embodiment, the therapeutically effective amount of the composition is of an amount of about 70 mg/kg body weight. According to another embodiment, the therapeutically effective amount of the composition is of an amount of about 80 mg/kg body weight. According to another embodiment, the therapeutically effective amount of the composition is of an amount of about 90 mg/kg body weight. According to another embodiment, the therapeutically effective amount of the composition is of an amount of about 100 mg/kg body weight. According to another embodiment, the therapeutically effective amount of the composition is of an amount of about 110 mg/kg body weight. According to another embodiment, the therapeutically effective amount of the composition is of an amount of about 120 mg/kg body weight. According to another embodiment, the therapeutically effective amount of the composition is of an amount of about 130 mg/kg body weight. According to another embodiment, the therapeutically effective amount of the composition is of an amount of about 140 mg/kg body weight. According to another embodiment, the therapeutically effective amount of the composition is of an amount of about 150 mg/kg body weight. According to another embodiment, the therapeutically effective amount of the composition is of an amount of about 160 mg/kg body weight. According to another embodiment, the therapeutically effective amount of the composition is of an amount of about 170 mg/kg body weight. According to another embodiment, the therapeutically effective amount of the composition is of an amount of about 180 mg/kg body weight. According to another embodiment, the therapeutically effective amount of the composition is of an amount of about 190 mg/kg body weight. According to another embodiment, the therapeutically effective amount of the composition is of an amount of about 200 mg/kg body weight. According to another embodiment, the therapeutically effective amount of the composition is of an amount of about 250 mg/kg body weight. According to another embodiment, the therapeutically effective amount of the composition is of an amount of about 500 mg/kg body weight.

The composition of the described invention may be presented conveniently in unit dosage form and may be prepared by any of the methods well known in the art of pharmacy. All methods include the step of bringing into association a therapeutic agent(s) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The compositions of the described invention may be administered in pharmaceutically acceptable solutions, which may routinely contain pharmaceutically acceptable concentrations of salt, buffering agents, preservatives, compatible carriers, adjuvants, and optionally other therapeutic ingredients.

According to some embodiments, the compositions of the described invention can further include one or more additional compatible active ingredients. The term “compatible” as used herein means that components of a composition are capable of being combined with each other in a manner such that there is no interaction that would substantially reduce the efficacy of the composition under ordinary use conditions. For example, without limitation, the Leptin, Leptin analog,s or derivative or Leptin agonist described herein may be mixed with other active materials that do not impair the desired action, or with materials that supplement the desired action.

Solutions or suspensions used for parenteral, intradermal, subcutaneous, subdural, intracerebral, intrathecal, or topical application may include, but are not limited to, for example, the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules (or ampules), disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

The sterile injectable preparation may also be a sterile injectable solution or suspension in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran.

In some embodiments, the compositions of the present invention may be formulated with an excipient or carrier including, but not limited to, a solvent. The terms “excipient” or “carrier” refer to substances that do not deleteriously react with the active compounds. Carriers must be of sufficiently high purity and of sufficiently low toxicity to render them suitable for administration to the subject being treated. The carrier can be inert, or it can possess pharmaceutical benefits.

The carrier can be liquid or solid and is selected with the planned manner of administration in mind to provide for the desired bulk, consistency, etc., when combined with an active and the other components of a given composition. Typical pharmaceutical carriers include, but are not limited to, binding agents (including, but not limited to pregelatinized maize starch, polyvinylpyrrolidone or hydroxypropyl methylcellulose); fillers (including but not limited to lactose and other sugars, microcrystalline cellulose, pectin, gelatin, calcium sulfate, ethyl cellulose, polyacrylates or calcium hydrogen phosphate.); lubricants (including, but not limited to magnesium stearate, talc, silica, sollidal silicon dioxide, stearic acid, metallic stearates, hydrogenated vegetable oils, corn starch, polyethylene glycols, sodium benzoate, sodium acetate); disintegrants (including but not limited to starch, sodium starch glycolate) and wetting agents (including but not limited to sodium lauryl sulfate). Additional suitable carriers for the compositions of the present invention include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds.

The term “pharmaceutically acceptable carrier” as used herein refers to any substantially non-toxic carrier conventionally useful for administration of pharmaceuticals in which the active component will remain stable and bioavailable. In some embodiments, the pharmaceutically acceptable carrier of the compositions of the present invention include a release agent such as a sustained release or delayed release carrier. In such embodiments, the carrier can be any material capable of sustained or delayed release of the active ingredient to provide a more efficient administration, resulting in less frequent and/or decreased dosage of the active ingredient, ease of handling, and extended or delayed effects.

A composition of the present invention, alone or in combination with other active ingredients, may be administered to a subject in a single dose or multiple doses over a period of time.

The pharmaceutical effect can be curing, minimizing, preventing or ameliorating a disease or disorder, or may have any other pharmaceutical beneficial effect. The concentration of the substance is selected so as to exert its or pharmaceutical effect, but low enough to avoid significant side effects within the scope and sound judgment of the physician. The effective amount of the composition may vary with the age and physical condition of the biological subject being treated, the severity of the condition, the duration of the treatment, the nature of concurrent therapy, the specific composition or other active ingredient employed, the particular carrier utilized, and like factors.

The concentration of the active in the compositions is selected so as to exert its therapeutic effect, but low enough to avoid significant side effects within the scope and sound judgment of the skilled artisan. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors. Those of skill in the art can readily evaluate such factors and, based on this information, determine the particular pharmaceutically effective amount of the compositions.

A skilled artisan can determine a pharmaceutically effective amount of the inventive compositions by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED50. The use of the term “individual” distinguishes the ED50 based on the intensity of effect as used herein from the median effective dose, also abbreviated ED50, determined from frequency of response data in a population. “Efficacy” as used herein refers to the property of the compositions of the present invention to achieve the desired response, and “maximum efficacy” refers to the maximum achievable effect. The amount of the compositions of the present invention that will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN′S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed also will depend on the route of administration, and the seriousness of the disease or disorder, and should be decided according to the judgment of the practitioner and each patient's circumstances. Various administration patterns will be apparent to those skilled in the art.

Those skilled in the art will recognize that initial indications of the appropriate therapeutic dosage of the compositions of the invention can be determined in in vitro and in vivo animal model systems, and in human clinical trials. One of skill in the art would know to use animal studies and human experience to identify a dosage that can safely be administered without generating toxicity or other side effects. For acute treatment, it is preferred that the therapeutic dosage be close to the maximum tolerated dose. For chronic preventive use, lower dosages may be desirable because of concerns about long term effects. Additional compositions of the present invention can be readily prepared using technology which is known in the art such as described in Remington's Pharmaceutical Sciences, 18th or 19th editions, published by the Mack Publishing Company of Easton, Pennsylvania, which is incorporated herein by reference.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Reagents and Antibodies

Minimum essential medium (MEM) was purchased from ATCC (Manassas, Va.). Fetal bovine serum (FBS), all-trans retinoic acid, nicotinamide and recombinant human Leptin were purchased from Sigma-Aldrich (St. Louis, Mo.). Compound C was purchased from EMD Biosciences (San Diego, Calif.). Rabbit anti-tau (pThr¹⁸¹) was purchased from Abcam (Cambridge, Mass.). Tau (tau46) mAb was purchased from Cell Signaling.

### Culture and Stable Transfection of Cell Lines

The human neuroblastoma cell line, SH-SY5Y, was purchased from American Type Culture Collection (ATCC). Cell culture was performed according to manufacturer's specific guidelines.

Briefly, cells were propagated in Eagle's Minimum Essential Medium (MEM) containing 10% Fetal Bovine Serum (FBS) until 80% -90% confluence; detached from the flask by Trypsin-EDTA; and then subcultured at a ratio of 1:5.

For neuronal differentiation, 1×10⁶ SY5Y or IMR-32 cells were grown in neruonal induction medium (NIM), which is consisted of MEM containing 2% FBS supplemented with 10 μM retinoic acid (RA). Cells were incubated in NIM for 6 days, and switched to serum-free NIM prior to treatment and harvesting on day 7.

To generate SY5Y stably over-expressing amyloid precursor protein (APP), cells were transfected with a mammalian expression vector encoding the 751 amino acid isoform of human APP (APP₇₅₁-Accession #NM_{—}201413) (Origene Technologies; Rockville, Md.) using the FuGENE HD transfection reagent, according to manufacturer's specific instructions (Promega; Madison, Wis.). Briefly, cells were transiently transfected with APP751 or vehicle for 48 h and then switched into selection medium containing a concentration range of the antibiotic G418 (100-600 μg/mL) to determine the optimal dose for stable selection. Selection media was changed every 3 days to remove non-viable cells. After 3 weeks, 200 μg/mL G418 yielded distinct colonies while all vehicle-transfected cells were non-viable. Cells were maintained in 10% FBS media containing 200 μg/mL G418 for expansion.

### Protein Extraction and Immunoblotting

Neuronal cells were treated with Leptin (500 ng/ml) in the presence or absence of nicotinamide (5 mM) or compound C (20 μM) for 6 or 18 h, depending on readout, and then harvested by scraping. Preparation of lysates and immunoblotting were performed as described.

### AMP-Activated Protein Kinase (AMPK) Activity Assay

AMP-Activated Protein Kinase (AMPK) activity in cell lysates was determined using the CycLex AMPK Kinase Assay Kit (MBL International; Woburn, Mass.), as described previously. Briefly, the term “relative AMPK activity” or “AMPK activity,” is defined as Compound C-sensitive protein kinase activity in cell lysates. Titration of various Compound C doses identified 10 μM as the dose in which there was no further reduction in kinase activity upon increasing concentration (data not shown). Neuronal cell lysates were incubated in the presence or absence of 10 μM Compound C, and protein kinase activity determined by measuring phosphorylation of the Insulin Receptor Substrate-1 (IRS-1) through immunoassay and conversion of a chromogenic substrate at an absorbance of 450 nm (A₄₅₀). Normalized AMPK activity in the lysates was defined as:

[A450−A450_{(+compound C)}]/μg protein×10³

### SIRT Activity Assay

Total Sirtuins activity in cell lysates was determined using the Fluro-de-Lys® HDAC Fluorimetric Cellular Activity Assay Kit (Enzo Life Sciences; Plymouth Meeting, Pa.), according to manufacturer's specified guidelines. “SIRT activity” in this assay is defined as nicotinamide-sensitive deacetylase (class III HDAC) activity in cell lysates. Briefly, 5 mM nicotinamide was identified by the manufacturer as a dose in which there was no further reduction in deacetylase activity upon increasing concentration. Neuronal cell lysates were incubated in the presence or absence of 5 mM nicotinamide, and SIRT activity determined by adding the Flour-de-Lys® Substrate for deacetylation followed by exposure to the Fluor-de-Lys® Developer to generate a fluorescent signal for detection using a fluorimeter (Ex. 350-380 nm; Em. 440-460 nm). Normalized SIRT activity in the lysates was measured in units of fluorescence intensity (Fᵢ) and defined as:

[Fi−Fi_{(+nicotinamide)}]/μg protein

### Enzyme-Linked Immunosorbent Assays (ELISAs)

Aβ₍₁₋₄₀₎ levels in cell culture media were determined using the Human β-Amyloid 1-40 ELISA kit (Invitrogen; Carlsbad, Calif.), and phospho- and total tau levels in cell culture lysates were determined using the Human Tau pSer³⁹⁶, pThr²³¹ and Total Tau ELISA kits (Invitrogen) according to manufacturer's specific instructions.

The Human β-Amyloid 1-40 ELISA kit was used for the quantitative determination of human Aβ40 in samples (e.g., tissue culture medium, tissue homogenate, cerebrospinal fluid (CSF), and the like). The assay recognizes both natural and synthetic forms of human Aβ40. The anti-human Aβ40 antibody is capable of selectively detecting Aβ40 and not Aβ42/Aβ43. The Human β-Amyloid 1-40 ELISA kit is a solid phase sandwich Enzyme Linked-Immuno-Sorbent Assay (ELISA). During the first incubation, standards of known human Aβ40 content, controls, and unknown samples are pipetted into the wells of the mcrotiter strips, which are coated with a monoclonal antibody specific for the NH₂-terminus of human Aβ, and co-incubated with a rabbit antibody specific for the COOH-terminus of the 1-40 Aβ sequence. This COOH-terminal sequence is created upon cleavage of the analyzed precursor. Bound rabbit antibody then is detected by the use of a horseradish peroxidase (HRP)-labeled anti-rabbit antibody. After washing, horseradish peroxidase (HRP)-labeled anti-rabbit antibody (enzyme) is added. After a second incubation and washing to remove the entire unbound enzyme, a substrate solution is added, which is acted upon by the bound enzyme to produce color. The intensity of this colored product is directly proportional to the concentration of human Aβ40 present in the original specimen.

The Invitrogen Human Tau (Total) kit is a solid phase sandwich Enzyme Linked-Immuno-Sorbent Assay (ELISA). Samples, including standards of known human Tau content, control specimens, and unknowns, are pipetted into the wells of microtiter strips, which are coated with a monoclonal antibody specific for human Tau. During the first incubation, the human Tau antigen binds to the immobilized (capture) antibody on one site. After washing, a rabbit polyclonal antibody specific for human Tau is added. During the second incubation, this antibody binds to the immobilized human Tau captured during the first incubation. After removal of excess second antibody, a horseradish peroxidase (HRP)-labeled anti-rabbit antibody is added. This binds to the rabbit polyclonal antibody to complete the four-member sandwich. After a third incubation and washing to remove all the excess anti-rabbit HRP, a substrate solution is added, which is acted upon by the bound enzyme to produce color. The intensity of this colored product is directly proportional to the concentration of human Tau present in the original specimen.

### Statistical Analyses

Statistical data analyses were performed with analysis of variance and Tukey-Kramer multiple comparisons test. Densitometric analyses were performed using the UN-SCAN-IT gel 6.1 software (Silk Scientific; Orem, UT). p<0.05 was considered statistically significant.

### Example 1

### Leptin Activates AMPK and SIRT in Neuronal Cells

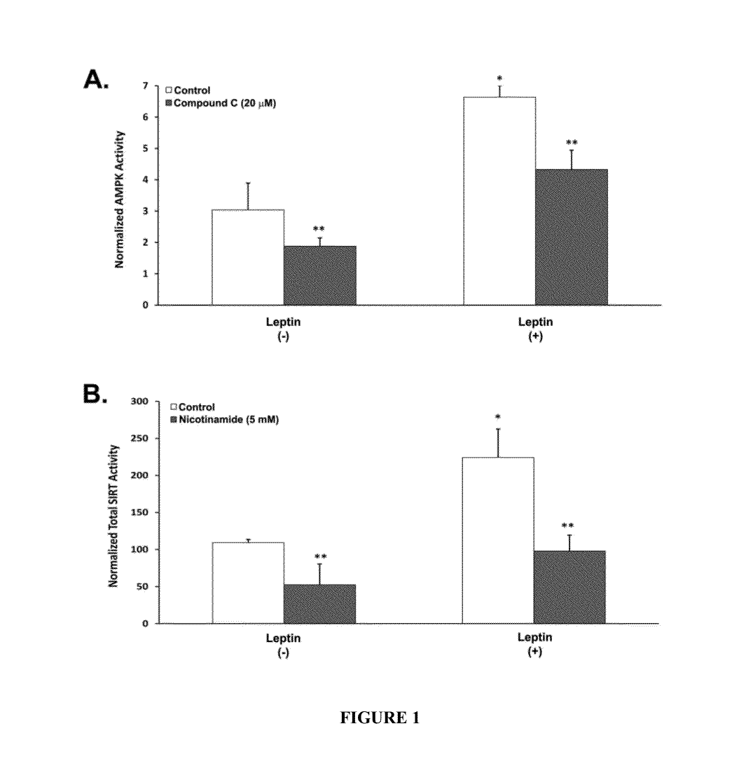

Whether treatment of neuronal cells with Leptin can boost cellular metabolism by directly increasing AMPK kinase activity and the total Sirtuin (SIRT) deacetylase activity was investigated (FIG. 1). Previous studies have shown that Leptin induces phosphorylation of AMPK in neurons, but have not determined its effects on AMPK kinase activity. SY5Y neuroblastoma cells were differentiated with retinoic acid (RA-SY5Y) and then treated with Leptin for 6 h in the presence or absence of the AMPK inhibitor, compound C, and AMPK activity measured. Leptin produced a significant increase in kinase activity (p<0.05) that was approximately 2-fold greater than in cells treated without Leptin (FIG. 1A, white bars). The increase in kinase activity was attenuated by co-treatment with compound C, which significantly (p<0.05) reduced AMPK activity (right gray bar) to levels similar to the non-treated control group (left white bar).

Leptin has been reported to increase gene expression of SIRT-1 in models of cerebral ischemia, yet no studies to date have ascribed an ability of Leptin to increase SIRT deacetylase activity in neurons. Cells treated with Leptin for 6 h showed a significant (p<0.05) increase in total SIRT activity that was approximately 2.5-fold greater than in cells treated without Leptin (FIG. 1B, white bars). The increase in deacetylase activity was blunted by co-treatment with the SIRT inhibitor, nicotinamide, which significantly (p<0.05) reduced SIRT activity (right gray bar) to levels similar to the non-treated control group (left white bar). These results suggest that Leptin can directly stimulate cellular metabolism in neurons through activation of endogenous energy sensors.

### Example 2

### Leptin's Effects on Tau Phosphorylation and β-Amyloid are Dependent on SIRT and AMPK

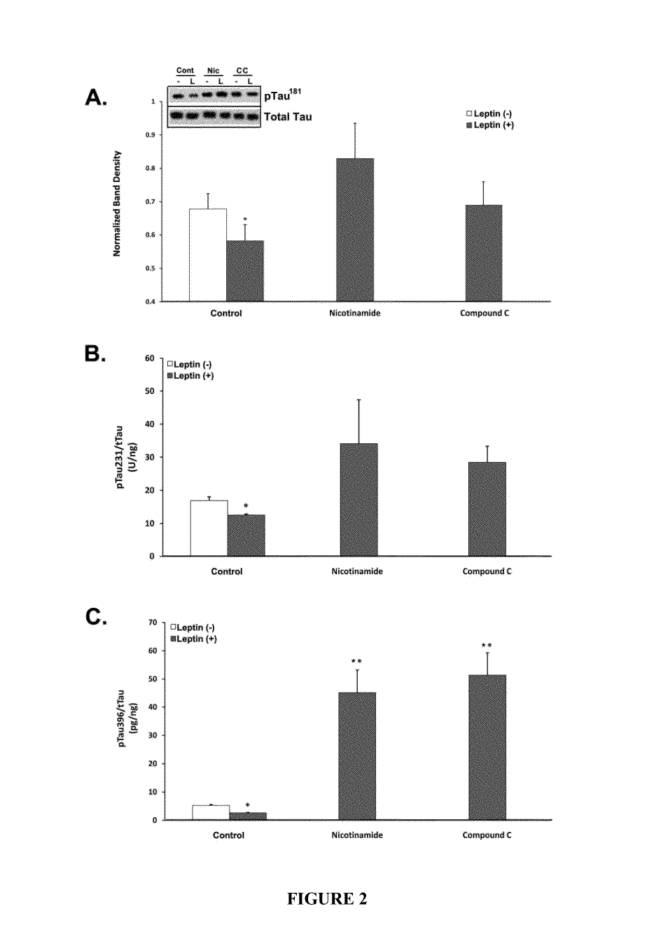

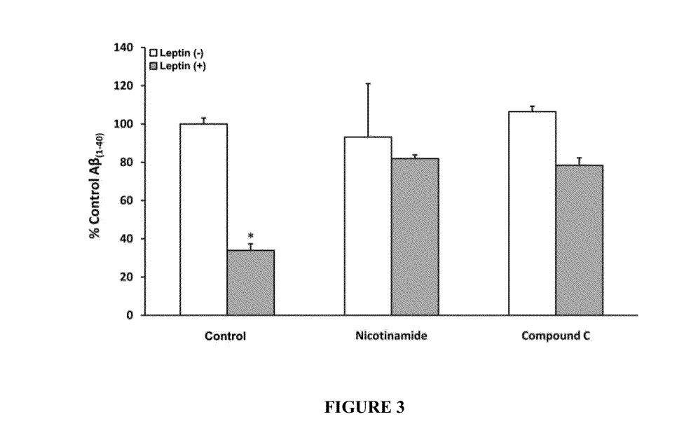

Whether modulating cellular energy metabolism through activation of Sirtuins (SIRT) and 5′-Adenosine Monophosphate Activated Protein Kinase (AMPK) mediates Leptin's reducing effects on tau phosphorylation (FIGS. 2) and β-amyloid production was analyzed (FIG. 3).

First, the Leptin's effects on tau phosphorylation was examined by utilizing antibodies against three different phospho-epitopes of tau via either immunoblot or enzyme-linked immunosorbent assay (ELISA) (FIG. 2): pTau¹⁸¹ (an Alzheimer's disease cerebrospinal fluid (CSF) clinical biomarker) (FIG. 2A); pTau²³¹ (phosphoepitope within the microtubule-binding domain of tau and involved in microtubule destabilization) (FIG. 2B); and pTau³⁹⁶ (implicated in oligomeric tau formation)(FIG. 2C). The levels of these specific phosphorylated tau forms were measured in RA-SY5Y cells following treatment with Leptin for 6 h, in the presence or absence of nicotinamide (middle group) or compound C (right group). Leptin-treated cells in the absence of SIRT or AMPK inhibition showed a significant (p<0.05) reduction in all examined phospho-tau species (left gray bars). However, co-treatment with either inhibitor negated these effects and returned phospho-tau to levels observed for the control group (left white bars). Inhibition of SIRT or AMPK significantly (p<0.05) increased Tau³⁹⁶ phosphorylation compared to control, independent of Leptin treatment (FIG. 2C).

Next, whether inhibition of SIRT or AMPK also can blunt Leptin's ability to impede production of β-amyloid in neurons (FIG. 3) was examined. SY5Y stably expressing human APP₇₅₁ (SY5Y-APP₇₅₁) were treated with Leptin for 18 h in the presence or absence of nicotinamide (middle group) or compound C (far right group) and the amount of Aβ₍₁₋₄₀₎ present in culture media was measured by ELISAs. Similar to the results observed for phosphorylation of tau (FIG. 2), Leptin-treated cells in the absence of inhibitors showed a significant (p<0.05) reduction in Aβ₍₁₋₄₀₎ (left gray bar), while co-treatment with either inhibitor (middle and right gray bars) reversed these effects and returned Aβ₍₁₋₄₀₎ to levels observed for the vehicle group (left white bar).

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

What is claimed is:

1. A method for enhancing an enzymatic activity of at least one family member of Sirtuins (SIRT) in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to enhance the enzymatic activity of at least one family member of Sirtuins (SIRT) in the neuronal cell population.

2. The method according to claim 1, wherein the Leptin analog or derivative is a functional analog of Leptin that is capable of binding to a Leptin receptor (OB—R) and of inducing a signal transduction pathway via the Leptin receptor (OB—R) inside at least one neuronal cell in the neuronal cell population.

3. The method according to claim 1, wherein the Leptin analog or derivative is selected from the group consisting of adiponectin, LY396623, Metreleptin, a murine Leptin analog, pegylated Leptin, methionyl human Leptin, Resistin, and a combination thereof.

4. The method according to claim 1, wherein the Leptin analog or derivative is a peptide or polypeptide in which at least one amino acid residue of Leptin has been replaced with at least one non-naturally occurring amino acid selected from the group consisting of beta-alanine, alpha amino butyric acid, gamma-amino butyric acid, alpha-isobutryic acid, norvaline, norleucine, epsilon-lysine, ornithine, homoserine, and hydroxyproline.

5. The method according to claim 1, wherein the Leptin agonist is an activator of AMP-activated protein kinase (AMPK).

6. The method according to claim 5, wherein the activator of AMP-activated protein kinase (AMPK) is selected from the group consisting of phenformin, 5-aminoimidazole-4-carboxamide riboside (AICAR), metformin, rosiglitazone, and a combination thereof.

7. The method according to claim 1, wherein contacting the neuronal cell population comprises administering the composition comprising the effective amount of the Leptin, the Leptin analog or derivative, or the Leptin agonist, and a carrier to a mammal in vivo.

8. The method according to claim 1, wherein contacting the neuronal cell population with the composition enhances an enzymatic activity of at least one family member of Sirtuins or AMP-activated Protein Kinase (AMPK) in the neuronal cell population compared to a control neuron population without treatment.

9. The method according to claim 1, wherein the family member of Sirtuins is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

10. The method according to claim 1, wherein the enzymatic activity of total Sirtuins in the neuronal cell population treated with the composition is at least two times greater than the enzymatic activity of total Sirtuins in a control neuron population without treatment.

11. The method according to claim 1, wherein the neuronal cell population comprises a neuronal population of central nervous system that expresses Obese Receptor (Ob—R).

12. The method according to claim 11, wherein the Obese Receptor is Obese receptor-Rb (Ob—Rb).

13. The method according to claim 1, wherein the neuronal cell population comprises a hippocampal neuron population.

14. The method according to claim 1, wherein the neuronal cell population comprises a cortical neuron population.

15. The method according to claim 1, wherein the neuronal cell population comprises a Purkinje neuron population.

16. The method according to claim 1, wherein the neuronal cell population comprises a basal ganglia neuron population.

17. The method according to claim 1 wherein the neuronal cell population comprises an olfactory neuron population.

18. The method according to claim 1, wherein the neuronal cell population comprises a dopaminergic neuron population.

19. The method according to claim 1, wherein the neuronal cell population comprises a noradrenergic neuron population.

20. The method according to claim 1, wherein the neuronal cell population comprises a motor neuron population.

21. The method according to claim 20, wherein the motor neuron population comprises a spinal motor neuron population.

22. The method according to claim 1, wherein the neuronal cell population comprises a sensory neuron population.

23. The method according to claim 1, wherein the neuronal cell population comprises an interneuron population.

24. The method according to claim 1, wherein the neuronal cell population comprises a neuronal population of peripheral nervous system.

25. A method for delaying senescence or cell death of a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is to effective to delay senescence or cell death of the neuronal cell population.

26. The method according to claim 25, wherein the Leptin analog or derivative is a functional analog of Leptin, which is capable of binding to a Leptin receptor (OB—R) and inducing a signal transduction pathway via the Leptin receptor (OB—R) inside at least one neuronal cell in the neuronal cell population.

27. The method according to claim 25, wherein the Leptin analog or derivative is selected from the group consisting of adiponectin, LY396623, Metreleptin, a murine Leptin analog, pegylated Leptin, methionyl human Leptin, Resistin, and a combination thereof.

28. The method according to claim 25, wherein the Leptin analog or derivative is a peptide or polypeptide in which at least one amino acid residue of Leptin has been replaced with non-naturally occurring amino acids selected from the group consisting of beta-alanine, alpha amino butyric acid, gamma-amino butyric acid, alpha-isobutryic acid, norvaline, norleucine, epsilon-lysine, ornithine, homoserine, and hydroxyproline.

29. The method according to claim 25, wherein the Leptin agonist is an activator of AMP-activated protein kinase (AMPK).

30. The method according to claim 29, wherein the activator of AMP-activated protein kinase (AMPK) is selected from the group consisting of phenformin, 5-aminoimidazole-4-carboxamide riboside (AICAR), metformin, rosiglitazone, and a combination thereof.

31. The method according to claim 25, wherein contacting the neuronal cell population comprises administering the composition comprising the effective amount of the Leptin, the Leptin analog or derivative, or the Leptin agonist, and the carrier to a mammal in vivo.

32. The method according to claim 25, wherein contacting the neuronal cell population with the composition enhances an enzymatic activity of at least one family member of Sirtuins or AMP-activated Protein Kinase (AMPK) in the neuronal cell population compared to a control neuron population without treatment.

33. The method according to claim 32, wherein the family member of Sirtuins is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

34. The method according to claim 32, wherein an enzymatic activity of total Sirtuins in the neuronal cell population treated with the composition is at least two times greater than the enzymatic activity of total Sirtuins in a control neuron population without treatment.

35. The method according to claim 32, wherein the enzymatic activity of the AMP-activated Protein Kinase (AMPK) in the neuronal cell population treated with the composition is at least two times greater than the enzymatic activity of the AMP-activated Protein Kinase (AMPK) in a control neuron population without treatment.

36. The method according to claim 25, wherein the neuronal cell population comprises a neuronal population of central nervous system that expresses Obese Receptor (OB—R).

37. The method according to claim 36, wherein the Obese Receptor is Obese Receptor-Rb (Ob—Rb).

38. The method according to claim 25, wherein the neuronal cell population comprises a hippocampal neuron population.

39. The method according to claim 25, wherein the neuronal cell population comprises a cortical neuron population.

40. The method according to claim 25, wherein the neuronal cell population comprises a Purkinje neuron population.

41. The method according to claim 25, wherein the neuronal cell population comprises a basal ganglia neuron population.

42. The method according to claim 25, wherein the neuronal cell population comprises an olfactory neuron population.

43. The method according to claim 25, wherein the neuronal cell population comprises a dopaminergic neuron population.

44. The method according to claim 25, wherein the neuronal cell population comprises a noradrenergic neuron population.

45. The method according to claim 25, wherein the neuronal cell population comprises a motor neuron population.

46. The method according to claim 45, wherein the motor neuron population comprises a spinal motor neuron population.

47. The method according to claim 25, wherein the neuronal cell population comprises a sensory neuron population.

48. The method according to claim 25, wherein the neuronal cell population comprises an interneuron population.

49. The method according to claim 25, wherein the neuronal cell population comprises a neuronal population of peripheral nervous system.
